(19) United States
(12) Patent Application Publication
(10) Pub. No.: US 2026/0151642 A1
(43) Pub. Date: Jun. 4, 2026

(54) Medical Clothing And Method For Treating Headaches And Migraines

(71) Applicant: Incrediwear Holdings, Inc., Chico, CA (US)

(72) Inventor: Jackson Corley, Chico, CA (US)

(73) Assignee: Incrediwear Holdings, Inc., Chico, CA (US)

(21) Appl. No.: 19/459,043

(22) Filed: Jan. 26, 2026

Related U.S. Application Data
(63) Continuation-in-part of application No. 18/942,882, filed on Nov. 11, 2024.
Application No. 18/942,882 is a continuation-in-part of application No. 18/451,793, filed on Aug. 17, 2023, now Pat. No. 12,186,552.
(60) Provisional application No. 63/387,453, filed on Dec. 14, 2022.
(60) Provisional application No. 63/387,486, filed on Dec. 14, 2022.
(60) Provisional application No. 63/754,070, filed on Feb. 5, 2025.

(51) Int. Cl. A61N 2/00; A61N 5/06

(52) U.S. Cl. CPC A61N 2/008; A61N 2/002; A61N 2/006; A61N 5/06; A61N 2005/0632; A61N 2005/0647; A61N 2005/065; A61N 2005/0659

(57) ABSTRACT

Medical clothing for treating headaches and migraines in a human includes a woven structure of threads including emissive threads having integrated semiconductor nanoparticles and carbon nanoparticles. The clothing is configured to be worn in contact with the head, following the contour of the head, but without compression of the head. In addition, the clothing is configured to place one or more of the emissive threads in proximity to peripheral nerves and neural pathways in the head. A method for treating headaches and migraines in a human includes providing clothing made from emissive threads having integrated semiconductor nanoparticles and carbon nanoparticles, and wearing the clothing on the head with a woven structure of the clothing in contact with the head without compression and with the emissive threads in proximity to peripheral nerves and neural pathways in the head.

### CROSS REFERENCE TO RELATED APPLICATIONS

This application is a continuation-in-part of application Ser. No. 18/942,882, filed Nov. 11, 2024, which is a continuation-in-part of application Ser. No. 18/451,793, filed Aug. 17, 2023, U.S. Pat. No. 12,186,552, which claims benefit of provisional application Ser. No. 63/387,453, filed Dec. 14, 2022, and claims benefit of provisional application Ser. No. 63/387,486, filed Dec. 14, 2022. This application also claims the benefit of provisional application Ser. No. 63/754,070, filed Feb. 5, 2025. All of these parent applications are incorporated herein by reference.

### FIELD

This disclosure relates to medical clothing and to a method for treating headaches and migraines in a human using the clothing.

### BACKGROUND

One type of headache is known as migraine. Migraines are one of the most common neurological diseases and ranks as the sixth cause of disability in the world. Currently available oral pharmacological treatments for migraines can lead to adverse effects, such as acute medication overuse and low efficacy. The present disclosure is directed to medical clothing configured to provide a treatment option for headaches and migraine that does not interfere other treatment protocols.

### SUMMARY

Medical clothing for treating headaches and migraines in a human includes a woven structure of threads including emissive threads having integrated semiconductor nanoparticles and carbon nanoparticles. In a first embodiment, each emissive thread comprises a composite thread having at least three components in an integrated structure, including a functional material, the semiconductor nanoparticles compounded with the functional material, and the carbon nanoparticles compounded with the functional material. In a second embodiment, each emissive thread comprises a twisted thread formed by at least three separate threads including: a base thread, a semiconductor thread containing the semiconductor nanoparticles, and a carbon thread containing the carbon nanoparticles, twisted together into a yarn. In both embodiments, one or more functional threads, such as cotton, nylon, polyester or jade threads, can also be incorporated into the woven structure.

The woven structure is configured to be worn in contact with the head, following a topography of the head, but without compression of the head. In addition, the woven structure is configured to place one or more of the emissive threads in proximity to peripheral nerves and neural pathways in the head. Exemplary configurations for the woven structure include headbands and hats. When the woven structure of the clothing contacts the head, the semiconductor nanoparticles are activated to emit negative ions responsive to a first transdermal effect from body heat flowing through the skin into the emissive threads for generating a micro electromagnetic field proximate to the peripheral nerves and the neural pathways in the head. In addition, the carbon nanoparticles are also activated to emit infrared waves proximate to the peripheral nerves and the neural pathways in the head responsive to a second transdermal effect from the body heat flowing into the emissive threads.

The woven structure can be constructed to have selected areas that contain a high concentration of emissive threads in alignment with the brain and the peripheral nerves and neural pathways in the head. Further, a thread count and location of the emissive threads in the woven structure can be selected to achieve a desired location, intensity and focus for the micro electromagnetic field and the infrared waves. For example, the warp and weft of the woven structure of the clothing can be used to selectively incorporate emissive threads at different locations to focus the micro electromagnetic field and the infrared waves on different locations within the head. The infrared waves and negative ions are biologically active and have an intensity and focus sufficient to increase blood flow and blood velocity proximate to the brain and to the peripheral nerves and neural pathways in the head. In addition, the infrared waves and negative ions have an intensity and focus sufficient to mediate inflammatory and pain pathways in the body. These functions help to alleviate symptoms of migraine.

A method for treating headaches and migraines in a human includes the steps of providing clothing having a woven structure comprising emissive threads having integrated semiconductor nanoparticles and carbon nanoparticles; and wearing the clothing on a head of the human with the woven structure in contact with the head without compression and with the emissive threads in proximity to peripheral nerves and neural pathways in the head. The method also includes the step of releasing negative ions to form a micro electromagnetic field proximate to the peripheral nerves and neural pathways in the head using a first transdermal effect from body heat activating the semiconductor nanoparticles to release the negative ions and form the micro electromagnetic field. The method also includes the step of releasing infrared waves using a second transdermal effect from the body heat activating the carbon nanoparticles to release the infrared waves proximate to the peripheral nerves and neural pathways in the head.

### BRIEF DESCRIPTION OF THE DRAWINGS

All of the figures are schematic in nature and may not be drawn to scale.

FIG. 1 is a schematic perspective view illustrating medical clothing on a human having a head with a brain and peripheral nerves and neural pathways;

FIG. 2A is an enlarged schematic cross sectional view taken along section line 2A-2A of FIG. 1 illustrating a woven structure of the clothing;

FIG. 2B is an enlarged schematic cross sectional view taken along section line 2B-2B of FIG. 2A illustrating an emitting thread of the clothing along with a micro electromagnetic field and infrared waves;

FIG. 2C is an enlarged schematic cross sectional view taken along section line 2C-2C of FIG. 2A illustrating semiconductor nanoparticles and carbon nanoparticles in the emitting thread;

FIG. 2D is an enlarged schematic cross sectional view equivalent to FIG. 2C illustrating an alternate embodiment emitting thread;

FIG. 3 is a schematic diagram illustrating operational characteristics of the clothing in which negative ions and infrared waves are released responsive to body heat;

FIG. 4 is a schematic diagram illustrating operational characteristics of the clothing in which blood circulation in the head is increased; and

FIG. 5 is a flow diagram illustrating steps in a method for treating migraines in a human using clothing having semiconductor nanoparticles and carbon nanoparticles.

### DETAILED DESCRIPTION

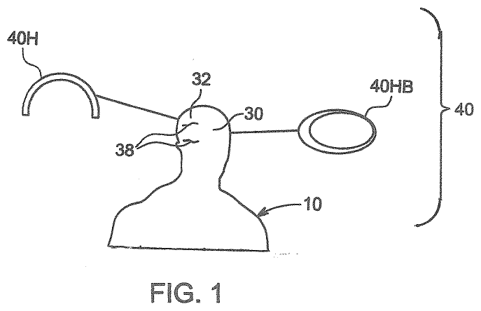

Referring to FIG. 1, a human 10 includes a head 32 and a brain 30 having peripheral nerves and neural pathways 38. As used herein “neural pathways” refer to interconnected networks of neurons in the brain 30 that transmit electrical signals between different parts of the nervous system.

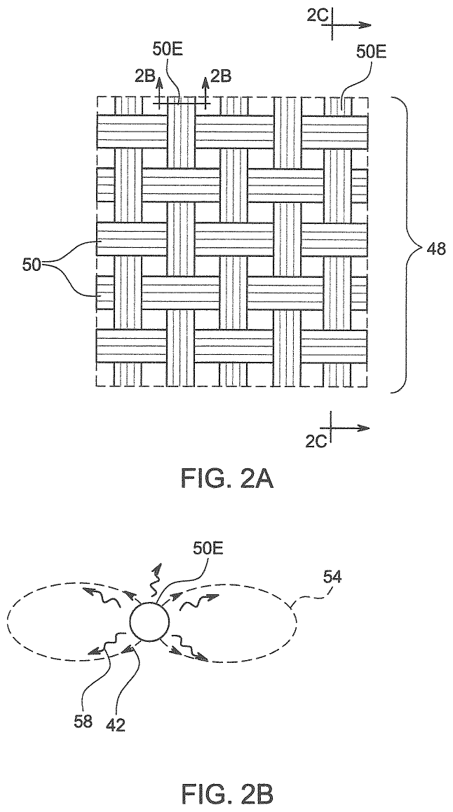

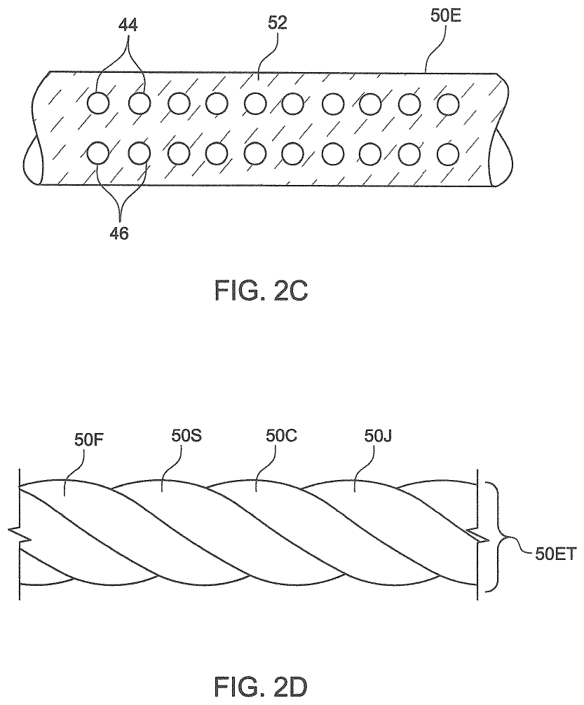

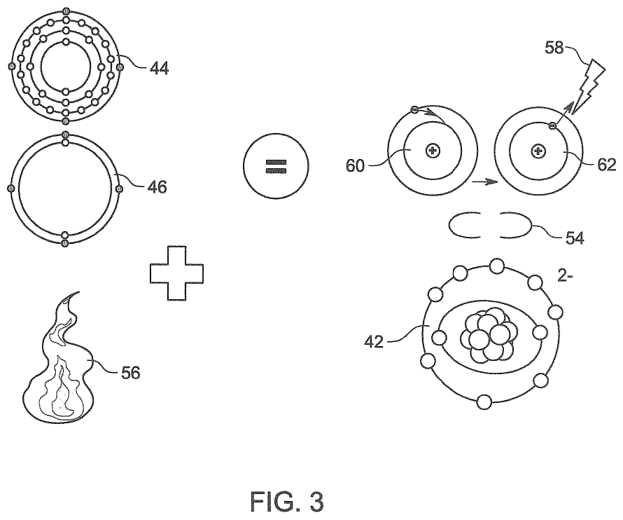

FIG. 1 illustrates medical clothing 40 for treating headaches and migraines. The clothing 40 can be constructed in a variety of clothing items including a hat 40H and a headband 40HB. However, it is to be understood that the hat 40H and a headband 40HB are exemplary, and other items of clothing can be configured to contact the head 32. In any case, the clothing 40 includes a woven structure 48 (FIG. 2A) comprising a plurality of threads 50 (FIG. 2A) including one or more emissive threads 50E (FIG. 2A) containing integrated semiconductor nanoparticles 44 (FIG. 2C) and integrated carbon nanoparticles 46 (FIG. 2C).

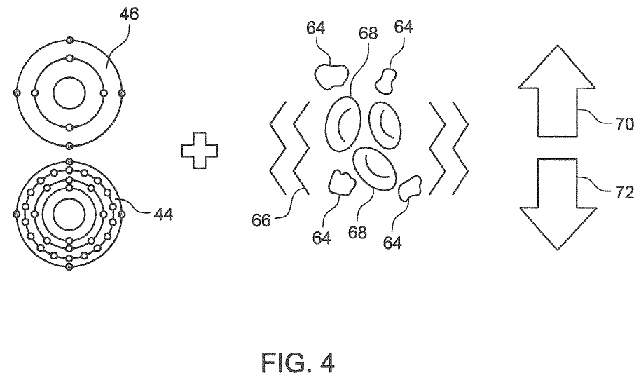

The clothing 40 can be formed entirely of the woven structure 48 (FIG. 2A) or can include only selected areas containing the woven structure 48 (FIG. 2A). The woven structure 48 (FIG. 2A) of the clothing 40 is designed to fit snuggly on the head 32 but without performing a compression function. In addition, the woven structure 48 (FIG. 2A) of the clothing 40 is designed to follow the topography of the head 32, and to stay in position on the head 32 during normal activity, with an inside surface of the woven structure 48 (FIG. 2A) of the clothing 40 in contact with the skin or scalp of the head 32. As will be further explained, the contact between the woven structure 48 (FIG. 2A) of the clothing 40 and the skin or scalp initiates transdermal effects in which body heat 56 (FIG. 3) activates the integrated semiconductor nanoparticles 44 (FIG. 2C) and carbon nanoparticles 46 (FIG. 2C).

Referring to FIGS. 2A, 2B and 2C, the woven structure 48 (FIG. 2A) of the clothing 40 (FIG. 1) includes a plurality of threads 50 woven together in any weaving pattern to form the woven structure 48. The woven structure 48 (FIG. 2A) includes a selected percentage of emissive threads 50E having integrated semiconductor nanoparticles 44 and carbon nanoparticles 46. By way of example, the selected percentage of the emissive threads 50E can be from 10% to 100% of the total number of threads 50, with between 10% to 30% considered a low concentration, between 30% to 70% considered a medium concentration, and between 70% to 100% considered a high concentration.

As shown in FIG. 2B, the emissive threads 50E emit negative ions 42 to form a micro electromagnetic field 54. As also shown in FIG. 2B, the emissive threads 50E also emit infrared waves 58. The woven structure 48 (FIG. 2A) can have high concentrations of the emissive threads 50E proximate to areas on the head 32 having peripheral nerves and neural pathways 38. For example, the areas proximate to the head temples (pterions) can include a high percentage of emissive threads 50E.

In addition, the woven structure 48 (FIG. 2A) of the clothing 40 can be shaped using techniques that are known in the art, such that the inside surface of the woven structure 48 (FIG. 2A) of the clothing 40 stays in contact with the head 32, and with the emissive threads 50E staying proximate to the peripheral nerves and neural pathways 38 (FIG. 1). Still further, by selectively incorporating emissive threads 50E in both the warp and weft of the woven structure 48, the peripheral nerves and neural pathways 38 can be surrounded by the emissive threads 50E, which can be used to focus the micro electromagnetic field 54 (FIG. 2B) and infrared waves 58 (FIG. 2B) on particular peripheral nerves and neural pathways 38 in the head 32 (FIG. 1).

In a first embodiment shown in FIG. 2C, each emissive thread 50E comprises a combination of at least three components in an integrated structure including: a functional material 52, a plurality of semiconductor nanoparticles 44 compounded with the functional material 52, and a plurality of carbon nanoparticles 46 compounded with the functional material 52. Exemplary materials for the functional material 52 include cotton, jade, nylon and polyester, either separately or in different combinations.

A preferred material for the semiconductor nanoparticles 44 comprises germanium (Ge) having a particle size on the order of 1000 nanometers (nm) or less. However, other semiconductor materials, such as silicon, silicon carbide, and selenium, as well as compound semiconductor materials, can also be used. A preferred material for the carbon nanoparticles 46 comprises charcoal (C), such as bamboo charcoal, having a particle size on the order of 1000 nanometers (nm) or less. Other equivalent forms of carbon can also be used to form the carbon nanoparticles 46.

The emissive thread 50E can be made using a process such as high temperature sintering (800-1000 degrees C.) of raw materials, such as germanium powder for the semiconductor nanoparticles 44 and carbonized charcoal for the carbon nanoparticles 46, followed by grinding and filtering into nanoparticles having a selected size range, with from 1 to 1000 nm being representative. The semiconductor nanoparticles 44 and the carbon nanoparticles 46 can then be compounded with the functional material 52 using a process such as polymerization or resin formation in essentially the same manufacturing process for synthetic threads.

In a second embodiment, each emissive thread 50ET comprises a plurality of twisted threads formed as a yarn including: a base thread 50F, a semiconductor thread 50S containing the semiconductor nanoparticles 44, and a carbon thread 50C containing the carbon nanoparticles 46 twisted together into a yarn. The emissive threads 50ET can also include other types of functional threads such as jade threads 50J and elastic threads (not shown).

Referring to FIG. 3, the semiconductor nanoparticles 44 are configured to release negative ions 42 responsive to a transdermal effect from body heat 56 in the head 32 contacted by the clothing 40 (FIG. 1). The semiconductor nanoparticles 44 have a concentration in the clothing 40 (FIG. 1) sufficient to generate a low intensity micro electromagnetic field 54. A representative intensity of the micro electromagnetic field 54 can be from 1730-2405 ions/cm³. One factor that affects the intensity of the micro electromagnetic field 54 is the concentration of the semiconductor nanoparticles 44 in the emissive thread 50E (and also in the twisted emissive thread 50ET). A representative concentration range of the semiconductor nanoparticles 44 as a weight % of the total weight of the emissive thread 50E can be from 5% to 80%. Another factor that affects the intensity of the micro electromagnetic field 54 is the thread count of the woven structure 48 (FIG. 2A) with a thread count of 50 to 600 being representative. In addition, the shape of the woven structure 48 (FIG. 2A) of the clothing 40 affects the intensity of the micro electromagnetic field 54, as the peripheral nerves and neural pathways 38 (FIG. 1) can be surrounded by the emissive threads 50E, which allows the intensity of the micro electromagnetic field 54 to be highest in areas proximate to the peripheral nerves and neural pathways 38 (FIG. 1).

As also shown in FIG. 3, the carbon nanoparticles 46 are configured to release infrared waves 58 responsive to the transdermal effect from the body heat 56 in the part of the head 32 contacted by the woven structure 48 (FIG. 2A) of the clothing 40. In FIG. 3, the infrared waves 58 are shown transitioning from a ground state 60 to an excited state 62 responsive to the body heat 56. A representative wavelength range of the infrared waves 58 can comprise mid infrared to far infrared, with from 1300 nm to 3000 nm being exemplary.

Referring to FIG. 4, operational characteristics of the clothing 40 are illustrated. In particular, the semiconductor nanoparticles 44 increase cellular vibration of body cells 64, 68 in the part of the head 32 contacted by the woven structure 48 (FIG. 2A) of the clothing 40, as indicated by the vibration waves 66. This increase in vibration of the body cells 64, 68 also increases blood circulation, and blood velocity, as indicated by up-arrow 70. At the same time, pain, inflammation and swelling decreases as indicated by down-arrow 72. In addition, the increased blood circulation provides O₂ and nutrients to the head 32 and reduces oxidative stress and free radicals. All of these functions that are initiated by wearing the clothing 40 help to alleviate the symptoms of migraine.

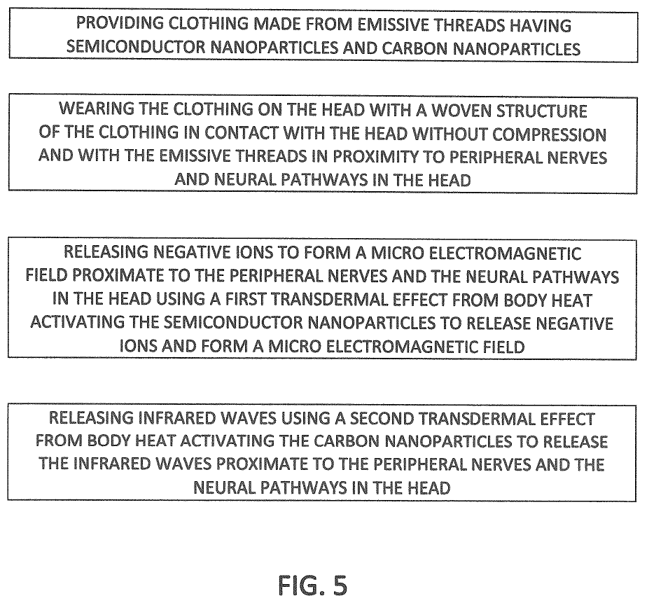

Referring to FIG. 5, steps in a method for treating headaches and migraine in a human are illustrated.

Example: To date, studies have shown that the medical clothing 40 increases circulation by up to 22% at rest, as well as reducing pain and inflammation. It is theorized that the increased blood flow, facilitates an anti-inflammatory nitric oxide (NO) cascade by accelerating the binding of calcium (Ca²⁺) to calmodulin (CaM). NO is known in the art to provide several healing factors to the body as a vasodilator, increasing blood and lymphatic flow. Additionally, NO down-regulates interleukin-1 beta (IL1β) and inducible nitric oxide synthase (iNOS) in certain cell types, which leads to reduced cyclooxygenase-2 (COX-2) and prostaglandins—molecules responsible for causing inflammation and pain. Unlike other systemic COX-2 inhibitors such as nonsteroidal anti-inflammatory drugs (NSAIDs), targeted infrared and negative ion therapy stimulate localized reaction pathways, thereby reducing pain and inflammation.

While a number of exemplary aspects and embodiments have been discussed above, those of skill in the art will recognize certain modifications, permutations, additions and subcombinations thereof. It is therefore intended that the following appended claims and claims hereafter introduced are interpreted to include all such modifications, permutations, additions and sub-combinations as are within their true spirit and scope.

What is claimed is:

1. Clothing for treating headaches and migraine in a human having a head with peripheral nerves and neural pathways comprising:
a woven structure comprising a plurality of threads including emissive threads having integrated semiconductor nanoparticles and carbon nanoparticles;
the woven structure configured to be worn in contact with the head, following a topography of the head, but without compression of the head, the woven structure configured to place one or more of the emissive threads in proximity to the peripheral nerves and the neural pathways in the head;
the semiconductor nanoparticles in the emissive threads configured to emit negative ions responsive to a first transdermal effect from body heat flowing into the emissive threads for generating a micro electromagnetic field proximate to the peripheral nerves and the neural pathways in the head; and
the carbon nanoparticles in the emissive threads configured to emit infrared waves proximate to the nerves and the neural pathways in the head responsive to a second transdermal effect from the body heat flowing into the emissive threads.

2. The clothing of claim 1 wherein each emissive thread comprises a composite thread comprising at least three components in an integrated structure, including a functional material, the semiconductor nanoparticles compounded with the functional material, and the carbon nanoparticles compounded with the functional material.

3. The clothing of claim 1 wherein each emissive thread comprises a twisted thread that includes at least three separate threads including a base thread, a semiconductor thread containing the semiconductor nanoparticles, and a carbon containing the carbon nanoparticles thread twisted together into a yarn.

4. The clothing of claim 1 wherein the woven structure includes one or more functional threads selected from the group consisting of cotton threads, nylon threads, polyester threads, and jade threads.

5. The clothing of claim 1 wherein a thread count and location of the emissive threads in the woven structure is selected to achieve a desired location, intensity and focus for the micro electromagnetic field and the infrared waves.

6. The clothing of claim 1 wherein the woven structure includes a selected percentage of the emissive threads in alignment with head temples of the head.

7. The clothing of claim 1 wherein the clothing comprises an item of clothing selected from the group consisting of headbands and hats.

8. The clothing of claim 1 wherein the infrared waves and negative ions have an intensity and focus sufficient to increase blood flow and blood velocity proximate to the peripheral nerves and neural pathways in the head.

9. The clothing of claim 1 wherein the infrared waves and negative ions have an intensity and focus sufficient to mediate inflammatory and pain pathways in the body.

10. The clothing of claim 1 wherein the semiconductor nanoparticles comprise germanium (Ge) having a particle size of 1000 nanometers (nm) or less.

11. The clothing of claim 1 wherein the carbon nanoparticles comprise charcoal (C), having a particle size of 1000 nanometers (nm) or less.

12. Clothing for treating headaches and migraine in a human with a head having peripheral nerves and neural pathways comprising:
a woven structure comprising a plurality of threads including emissive threads having integrated semiconductor nanoparticles and carbon nanoparticles;
the woven structure configured to be worn in contact with the head, following a topography of the head, but without compression of the head, the woven structure configured to place one or more of the emissive threads in proximity to the peripheral nerves and neural pathways in the head;
the semiconductor nanoparticles in the emissive threads configured to emit negative ions responsive to a first transdermal effect from body heat flowing into the emissive threads for generating a micro electromagnetic field proximate to the peripheral nerves and the neural pathways in the head; and
the carbon nanoparticles in the emissive threads configured to emit infrared waves proximate to the peripheral nerves and the neural pathways in the head responsive to a second transdermal effect from the body heat flowing into the emissive threads.

13. The clothing of claim 12 wherein each emissive thread comprises a composite thread comprising at least three components in an integrated structure, including a functional material, the semiconductor nanoparticles compounded with the functional material, and the carbon nanoparticles compounded with the functional material.

14. The clothing of claim 12 wherein each emissive thread comprises a twisted thread that includes at least three separate threads including a base thread, a semiconductor thread containing the semiconductor nanoparticles, and a carbon containing the carbon nanoparticles thread twisted together into a yarn.

15. The clothing of claim 12 wherein the woven structure includes one or more functional threads selected from the group consisting of cotton threads, nylon threads, polyester threads, and jade threads.

16. The clothing of claim 12 wherein a thread count and location of the emissive threads in the woven structure is selected to achieve a desired location, intensity and focus for the micro electromagnetic field and the infrared waves.

17. The clothing of claim 12 wherein the clothing comprises an item selected from the group consisting of headbands and hats.

18. A method for treating headaches and migraines in a human with a head having peripheral nerves and neural pathways comprising:
providing clothing having a woven structure comprising a plurality of emissive threads having integrated semiconductor nanoparticles and carbon nanoparticles;
wearing the clothing on the head with the woven structure in contact with the head without compression and with the emissive threads in proximity to the peripheral nerves and the neural pathways in the head;
releasing negative ions to form a micro electromagnetic field proximate to the peripheral nerves and the neural pathways in the head using a first transdermal effect from body heat activating the semiconductor nanoparticles to release the negative ions and form the micro electromagnetic field proximate to the peripheral nerves and the neural pathways in the head; and
releasing infrared waves using a second transdermal effect from the body heat activating the carbon nanoparticles to release the infrared waves proximate to the peripheral nerves and the neural pathways in the head.

19. The method of claim 18 wherein the clothing comprises an item selected from the group consisting of headbands and hats.

20. The method of claim 18 wherein a thread count and location of the emissive threads in the woven structure is selected to achieve a desired location, intensity and focus for the micro electromagnetic field and the infrared waves.
